(19) United States
(12) Patent Application Publication
(10) Pub. No.: US 2023/0119369 A1
(43) Pub. Date: Apr. 20, 2023

(54) CHEMICAL COMPOUND DATABASE CONSTRUCTION METHOD, COMPOSITION PREDICTION AND ASSEMBLING METHODS AND OBTAINED FRAGRANCES

(71) Applicant: Firmenich SA, Satigny (CH)

(72) Inventors: Vera TCHAKALOVA, Satigny (CH); Laura MESMIN, Satigny (CH)

(21) Appl. No.: 18/047,107

(22) Filed: Oct. 17, 2022

(30) Foreign Application Priority Data
Oct. 18, 2021 (EP) 21203197.5

(51) Int. Cl. G16C 20/62

(52) U.S. Cl. CPC G16C 20/62

(57) ABSTRACT

The computer implemented method (600) to provide predictive, real time, skin hydration performance metrics for a composition, comprises, at least:
a step (205) of selecting, upon a computer interface, at least one chemical compound digital identifier, to form a composition,
a step (610) of retrieving, from a database, at least one value representative of a polarity value of at least one selected chemical compound identifier,
a step (615) of predicting at least one moisturizing factor value for at least one chemical compound identifier or of the composition as a function of at least one retrieved polarity value and
a step (620) of outputting at least one moisturizing factor value predicted.

The invention also aims at volatile composition obtained by using the volatile composition assembling method of the present invention.

### TECHNICAL FIELD OF THE INVENTION

The present invention relates to a chemical compound physical parameter database construction method, a physical composition evolution prediction method to provide predictive, real-time, secondary cosmetic benefits volatile composition performance metrics and the corresponding systems. It applies, in particular, to the fields of fragrance and flavors, cosmetics, surface and body care, hygiene and pharmaceuticals.

### BACKGROUND OF THE INVENTION

Fragrance design can be defined as the selection of at least one fragrant ingredient to form a composition intended to provide a targeted fragrance. Fragrance design is most notably known in the field of perfumery and is performed by perfume designers.

The evaluation of a fragrance is typically based on fragrance performance metrics and fragrance hedonics alone. Several metrics are used today, such as the detectability of the fragrance by a human nose for example. Typically, such metrics fail to account for secondary benefits of fragrances, such as cosmetic benefits for example.

As such metrics lack for evaluating the performance of a fragrance according to said secondary benefits, no accurate performance predictor in these regards may be obtained. This leads to much wasted time in laboratories from inefficient but currently obligatory trial and error approaches.

Such issues are, however, not limited to the field of fragrance design but rather to all fields in which volatile chemicals are used and their performance evaluated on criteria limited to single benefits.

### SUMMARY OF THE INVENTION

The present invention is intended to remedy all or part of these disadvantages.

To this effect, according to a first aspect, the present invention aims at a computer implemented method to provide predictive, real time, skin hydration performance metrics for a composition, comprising, at least:
a step of selecting, upon a computer interface, at least one chemical compound digital identifier, to form a composition,
a step of retrieving, from a database, at least one value representative of a polarity value of at least one selected chemical compound identifier,
a step of predicting at least one moisturizing factor value for at least one chemical compound identifier or of the composition as a function of at least one so retrieved polarity value and
a step of outputting at least one moisturizing factor value predicted.

Such provisions allow for the constitution of a new technical composition performance indicator quantifying the capacity of a composition to participate in moisturizing the skin. Such an indicator can then be used in downstream computer systems and programs to quantify compounds and/or predict the capacity of a composition to participate in moisturizing the skin.

In this new approach, composition performance and cosmetic performance may be linked, which is not possible in other current known systems.

In particular embodiments, the comprises the method object of the present invention comprises a chemical compound physical parameter database construction step, comprising at least:
a step of controlled deposition of a volatile chemical compound, or a non-volatile compound in a liquid state, upon a skin replicating surface,
a step of measurement of a quantity of evaporated water from the skin replicating surface or of remaining water on the skin replicating surface after the deposition of the chemical compound at different measurement times,
a step of water evaporation rate calculation depending on the measured evaporated or remaining water quantities measured,
a step of moisturizing factor calculation as a function of the water evaporation rate calculated and
a step of storing, in a database, the calculated moisturizing factor and, optionally, the water evaporation rate calculated.

The construction of such a database allows for saving time in composition design and production processes by limiting the number of tests required to reach a target secondary benefit result.

In particular embodiments, the construction method object of the present invention further comprises:
a step of computing a chemical compound polarity for a chemical compound associated to a stored moisturizing factor,
a step of modeling of a mathematical formula of moisturizing factor as a function of chemical compound polarity and
a step of recording, in a database, the moisturizing factor formula modeled parameters.

This new approach links, for example, volatile compound polarity to moisturizing factor, representing moisturization capabilities for said volatile compound. Such provisions allow for the prediction of moisturization capabilities of any volatile chemical compound provided said compound is associated to data representative of associated compound polarity.

In particular embodiments, the modeled moisturizing factor is a function of a logarithm or exponential function of the chemical compound polarity.

Such embodiments allow for an accurate prediction of moisturizing factor as a function of chemical compound polarity.

In particular embodiments, the chemical compound polarity is computed as a function of at least one of the dispersion, polar and hydrogen bonding components of the cohesion energy density, related to the dispersion, polar and hydrogen bonding interactions of said chemical compound.

In particular embodiments, the method object of the present invention comprises a step of inputting, for each selected chemical compound, a quantity of said chemical compound, the step of obtaining comprising the steps of a step of computing a mean moisturizing factor for at least one chemical compound identifier as a function of the moisturizing factor retrieved and the quantity input for said chemical compound identifier, the step of outputting being configured to output at least one mean moisturizing factor computed.

Such provisions allow for the modeling of complex chemical compound interactions and the impact of one chemical compound on overall secondary benefits performance.

In particular embodiments, at least two chemical compound identifiers are selected, the method further comprising a step of computing a composition moisturizing factor as a function of at least two mean moisturizing factors computed.

Such provisions allow for the modeling of complex chemical compound interactions and the impact on overall secondary benefits performance. In particular embodiments, at least two chemical compound identifiers are selected, the method further comprising a step of computing a composition moisturizing factor as a function of at least two mean moisturizing factors predicted.

Such provisions allow for the modeling of complex chemical compound interactions and the impact of one chemical compound on overall secondary benefits performance.

In particular embodiments, at least two chemical compound identifiers are selected, the method further comprising a step of computing a moisturizing factor linearity of a composition of said at least two compound identifiers based on the obtained moisturizing factor of at least two selected chemical compound digital identifiers, the step of outputting being configured to display the moisturizing factor linearity of the composition of said at least two chemical compound digital identifiers.

Such provisions allow for the determination of the relative change in performance and composition of a particular composition formula. Linearity can represent either change in relative composition or change over time, in such a scenario.

In particular embodiments, the method object of the present invention further comprises a step of defining a moisturizing factor threshold, at least one chemical compound digital identifier being removed from the selection as a function of the difference between the moisturizing factor being obtained for said chemical compound digital identifier and the defined moisturizing factor threshold.

Such provisions allow for dynamic filtering of volatile chemical compounds to meet target secondary benefit performance criteria.

In particular embodiments, the method object of the present invention further comprises a step of replacing at least one chemical compound digital identifier as a function of the difference between the moisturizing factor being obtained for said chemical compound digital identifier and the moisturizing factor being obtained for an alternative chemical compound digital identifier candidate.

Such provisions allow for dynamic composition formula creation, wherein less performing chemical compounds, with regards to secondary benefits, are replaced by more performing chemical compounds. More complex embodiments may further use multi-criteria analysis to provide replacements for a target chemical compound.

According to a second aspect, the present invention aims at a chemical compound physical parameter database construction method, comprising, at least:
a step of measurement of a compound polarity,
a step of moisturizing factor calculation as a function of the measured compound polarity and
a step of storing, in a database, the calculated moisturizing factor and, optionally, the compound polarity measured.

Such provisions allow for the constitution of a new technical composition performance indicator quantifying the capacity of a composition to participate in moisturizing the skin. Such an indicator can then be used in downstream computer systems and programs to quantify volatile compounds and/or predict the capacity of a composition to participate in moisturizing the skin.

According to a third aspect, the present invention aims at a composition prediction method to provide predictive, real-time, secondary cosmetic benefits performance metrics, comprising, at least:
a step of selecting at least one chemical compound identifier upon a computerized interface,
a step of obtaining a moisturizing factor associated to at least one chemical compound identifier and
a step of outputting at least one mean moisturizing factor obtained.

Such provisions allow for the design of predictive composition design computer systems in which users may view predictive metrics for moisturizing performance of design compositions.

Such provisions further allow to:
speed up a composition (such as perfume) creation process,
optimizing the composition performance,
reformulating the composition performance easily,
new understanding to composition designers on their own formulas and
the comparison of formulas performance.

In particular embodiments, the step of obtaining comprises:
a step of retrieving, from a database, at least one value representative of the polarity of said chemical compound identifier,
a step of predicting at least one moisturizing factor for at least one chemical compound identifier as a function of the polarity of said chemical compound identifier
   the step of outputting being configured to output at least one moisturizing factor predicted.

Such provisions allow for the prediction of the moisturizing factor for a chemical compound based upon the bonding components of said chemical compound.

According to a fourth aspect, the present invention aims at a composition prediction method to provide predictive, real time, secondary cosmetic benefits performance metrics, characterized in that it comprises, at least:
a step of selecting, upon a computer interface, at least one chemical compound digital identifier, to form a composition,
a step of retrieving, from a database, at least one value representative of the moisturizing factor of at least one selected chemical compound identifier,
a step of predicting at least one polarity value for at least one chemical compound identifier or of the composition as a function of at least one retrieved moisturising factor and
a step of outputting at least one polarity value predicted.

Such dispositions allow for the reverse application of the mathematical formula linking moisturizing factor to polarity.

According to a fifth aspect, the present invention aims at a method of chemical compound composition assembly, comprising:
a composition prediction method object of the present invention and
a step of assembling the predicted object of the prediction method.

According to a sixth aspect, the present invention aims at a composition obtained according to an assembling method object of the present invention.

In particular embodiments, at least one chemical compound is a fragrant chemical compound.

According to a seventh aspect, the present invention aims at a volatile liquid chemical compound physical parameter database construction system, comprising, at least:
a means of controlled deposition of a volatile chemical compound, or a non-volatile compound in a liquid state, upon a skin replicating surface,
a means of measurement of a quantity of evaporated water from the skin replicating surface or of remaining water on the skin replicating surface after the deposition of the chemical compound at different measurement times,
a means of water evaporation rate calculation depending on the measured evaporated or remaining water quantities measured,
a means of moisturizing factor calculation as a function of the water evaporation rate calculated and
a means of storing, in a database, the calculated moisturizing factor and, optionally, the water evaporation rate calculated.

The advantages of this system are similar to the advantages of the corresponding methods.

According to an eighth aspect, the present invention aims at a composition evolution prediction system to provide predictive, real-time, secondary cosmetic benefits performance metrics, comprising, at least:
a means of selecting at least one chemical compound identifier upon a computerized interface,
a means of obtaining a moisturizing factor associated to at least one chemical compound identifier and
a means of outputting at least one mean moisturizing factor obtained.

The advantages of this system are similar to the advantages of the corresponding methods.

### BRIEF DESCRIPTION OF THE DRAWINGS

Other advantages, purposes and particular characteristics of the invention shall be apparent from the following non-exhaustive description of at least one particular method or system which is the subject of this invention, in relation to the drawings annexed hereto, in which:

FIG. 1 represents, schematically and in the form of a flowchart, a first particular succession of steps of the database construction method object of the present invention,

FIG. 2 represents, schematically and in the form of a flowchart, a particular succession of steps of the prediction method object of the present invention,

FIG. 3 represents, schematically, a particular embodiment of a system capable of implementing the database construction method object of the present invention,

FIG. 4 represents, schematically, a particular embodiment of a system capable of implementing the prediction method object of the present invention,

FIG. 5 represents, schematically, the result of a mathematical formula relating moisturizing factor of a chemical compound with the polarity of said chemical compound,

FIG. 6 represents, schematically and in the form of a flowchart, a particular succession of steps of the assembling method object of the present invention,

FIG. 7 represents, schematically and in the form of a flowchart, a particular succession of steps of the prediction method object of the present invention and

FIG. 8 represents, schematically and in the form of a flowchart, a second particular succession of steps of the database construction method object of the present invention.

### DETAILED DESCRIPTION OF THE INVENTION

This description is not exhaustive, as each feature of one embodiment may be combined with any other feature of any other embodiment in an advantageous manner.

It should be noted at this point that the figures are not to scale.

The description below is presented for the particular use-case of volatile chemical compounds parameter discovery and use. However, the description below may be understood as suited for the use-case of non-volatile chemical compounds parameter discovery and use.

As used herein, the terms “volatile chemical compound” designates any compound that evaporates in the air at environmental temperatures. Such compounds may designate pharmaceutical compounds or fragrant chemical compounds. In a non-limiting manner, the embodiments disclosed below aim at fragrant chemical compounds. The same embodiments could be adapted for pharmaceutical compounds or any other volatile chemical compound of interest.

As used herein, the terms “fragrant chemical compound” designates a perfuming ingredient, a flavor ingredient, a perfumery carrier, a flavor carrier, a perfumery adjuvant, a flavor adjuvant, a perfumery modulator, flavor modulator. Preferably, such a fragrance or fragrant chemical compound is volatile. Such an ingredient may be a natural ingredient.

By “perfuming ingredient” it is meant here a compound, which is used in a perfuming preparation or a composition to impart a hedonic effect. In other words, such an ingredient, to be considered as being a perfuming one, must be recognized by a person skilled in the art as being able to impart or modify in a positive or pleasant way the odor of a composition, and not just as having an odor.

The nature and type of the perfuming ingredients do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Said perfuming ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said perfuming ingredients may also be compounds known to release in a controlled manner various types of perfuming ingredients also known as properfume or profragrance.

By “perfumery carrier” it is meant here a material which is practically neutral from a perfumery point of view, i.e., that does not significantly alter the organoleptic properties of perfuming ingredients. Said carrier may be a liquid or a solid.

As liquid carrier one may cite, as non-limiting examples, an emulsifying system, i.e., a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethanol, tri-ethyl citrate or mixtures thereof, which are the most commonly used. For the compositions which comprise both a perfumery carrier and a perfumery base, other suitable perfumery carriers than those previously specified, can be also ethanol, water/ethanol mixtures, glycerol, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF), esters and emollients such as the trademark Cetiol®, vegetable oils, essential oils.

Solid carrier is meant to designate a material to which the perfuming composition or some element of the perfuming composition can be chemically or physically bound. In general, such solid carriers are employed either to stabilize the composition, or to control the rate of evaporation of the compositions or of some ingredients. The use of solid carriers is of current use in the art and a person skilled in the art knows how to reach the desired effect. However, by way of non-limiting examples of solid carriers, one may cite absorbing gums or polymers or inorganic materials, such as porous polymers, cyclodextrins, wood-based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualitat, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g., formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively, one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Others resins one are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine-based resins with aldehydes includes represented by articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is significant. More recent publications of pertinency, which disclose suitable uses of such microcapsules, are represented for example by the article of K. Bruyninckx and M. Dusselier, ACS Sustainable Chemistry & Engineering, 2019, vol. 7, pages 8041-8054H. Y. Lee et al. Journal of Microencapsulation, 2002, vol. 19, pages 559-569, international patent publication WO 01/41915 or yet the article of S. Bone et al. Chimia, 2011, vol. 65, pages 177-181.

By “perfumery adjuvant,” it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellents, ointments, vitamins and mixtures thereof.

By “perfumery modulator,” it is understood here an agent having the capacity to affect the manner in which the odor, and in particular the evaporation rate and intensity, of the compositions incorporating said modulator can be perceived by an observer or user thereof, over time, as compared to the same perception in the absence of the modulator. Perfumery modulators are also known as fixative. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10, isoceteth-12, isoceteth-15, isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and a combination thereof.

By “flavoring ingredient” it is meant here a compound, which is used in flavoring preparations or compositions to impart a hedonic effect. In other words, such an ingredient, to be considered as being a flavoring one, must be recognized by a person skilled in the art as being able to impart or modify in a positive or pleasant way the taste of a composition, and not just as having a taste. The nature and type of the flavoring ingredients present in the composition do not warrant a more detailed description here, the skilled person being able to select them on the basis of its general knowledge and according to intended use or application and the desired organoleptic effect. In general terms, these flavoring ingredients belong to chemical classes as varied as alcohols, aldehydes, ketones, esters, ethers, acetates, nitriles, terpenoids, nitrogenous or sulphurous heterocyclic compounds and essential oils, and said flavoring ingredients can be of natural or synthetic origin. Many of these ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of flavor. It is also understood that said co-ingredients may also be compounds known to release in a controlled manner various types of flavoring compounds, also called pro-flavor.

The term “flavor carrier” designates a material which is substantially neutral from a flavor point of view, as far as it does not significantly alter the organoleptic properties of flavoring ingredients. The carrier may be a liquid or a solid.

Suitable liquid carriers include, for instance, an emulsifying system, i.e., a solvent and a surfactant system, or a solvent commonly used in flavors. A detailed description of the nature and type of solvents commonly used in flavor cannot be exhaustive. Suitable solvents used in flavor include, for instance, propylene glycol, triacetine, caprylic/capric triglyceride (neobee®), triethyl citrate, benzylic alcohol, ethanol, isopropanol, citrus terpenes, vegetable oils such as Linseed oil, sunflower oil or coconut oil, glycerol.

Suitable solid carriers include, for instance, absorbing gums or polymers, or even encapsulating materials. Examples of such materials may comprise wall-forming and plasticizing materials, such as mono, di- or polysaccharides, natural or modified starches, hydrocolloids, cellulose derivatives, polyvinyl acetates, polyvinylalcohols, xanthan gum, arabic gum, acacia gum or yet the materials cited in reference texts such as H. Scherz, Hydrokolloid : Stabilisatoren, Dickungs-und Geliermittel in Lebensmitteln, Band 2 der Schriftenreihe Lebensmittelchemie, Lebensmittelqualitat, Behr's VerlagGmbH & Co., Hamburg, 1996. Encapsulation is a well-known process to a person skilled in the art, and may be performed, for instance, using techniques such as spray-drying, agglomeration, extrusion, coating, plating, coacervation and the like.

By “flavor adjuvant”, it is meant here an ingredient capable of imparting additional added benefit such as a color (e.g., caramel), chemical stability, and so on. A detailed description of the nature and type of adjuvant commonly used in flavoring compositions cannot be exhaustive. Nevertheless, such adjuvants are well known to a person skilled in the art who will be able to select them on the basis of its general knowledge and according to intended use or application. One may cite as specific non-limiting examples the following: viscosity agents (e.g., emulsifier, thickeners, gelling and/or rheology modifiers, e.g., pectin or agar gum), stabilizing agents (e.g., antioxidant, heat/light and or buffers agents e.g., citric acid), coloring agents (e.g., natural or synthetic or natural extract imparting color), preservatives (e.g., antibacterial or antimicrobial or antifungal agents, e.g., benzoic acid), vitamins and mixtures thereof.

By “flavor modulator,” it is meant here an ingredient capable to enhance sweetness, to block bitterness, to enhance umami, to reduce sourness or licorice taste, to enhance saltiness, to enhance a cooling effect, or any combinations of the foregoing. Flavor modulators are also called trigeminal sensates.

As used herein, the term “formula” designates a liquid, solid or gaseous assembly of at least one volatile molecule.

As used herein, the term “fragrance” refers to the olfactory perception resulting from the sum of odorant receptor activation, enhancement, and inhibition by at least one fragrant chemical compound.

As used herein, the terms “computing system” designate any electronic calculation device, whether unitary or distributed, capable of receiving numerical inputs and providing numerical outputs by and to any sort of interface, such as a digital interface. Typically, a computing system designates either a computer executing a software having access to data storage or a client-server architecture wherein the data and/or calculation is performed at the server side while the client side acts as an interface.

As used herein, the terms “digital identifier” refer to any computerized identifier, such as one used in a computer database, representing a physical object, such as a fragrant chemical compound.

In the context of this invention, a “skin replicating surface” designates any surface presenting physicochemical properties similar to the human skin, including human skin itself. Simple embodiments of such an artificial surface may focus on a mimicking a limited number of physicochemical properties, such as thickness, chemical reactivity, or visco-elasticity of the human skin. Other embodiments may focus on a particular element of the human skin, such as the epidermis, the dermis layer and/or stratum corneum of the human skin.

Such a surface may be as simple as a glass surface or as complex as a multi-layer model. The closer such surface is to replicating actual properties of the human skin, the better the quality of database construction and the better the quality of downstream prediction.

In the context of this invention, “moisturizing factor” is defined as a relative change of the TEWL (transepidermal water loss) after the skin treatment with the moisturizing compound compared to the TEWL of the skin before the product deposition.

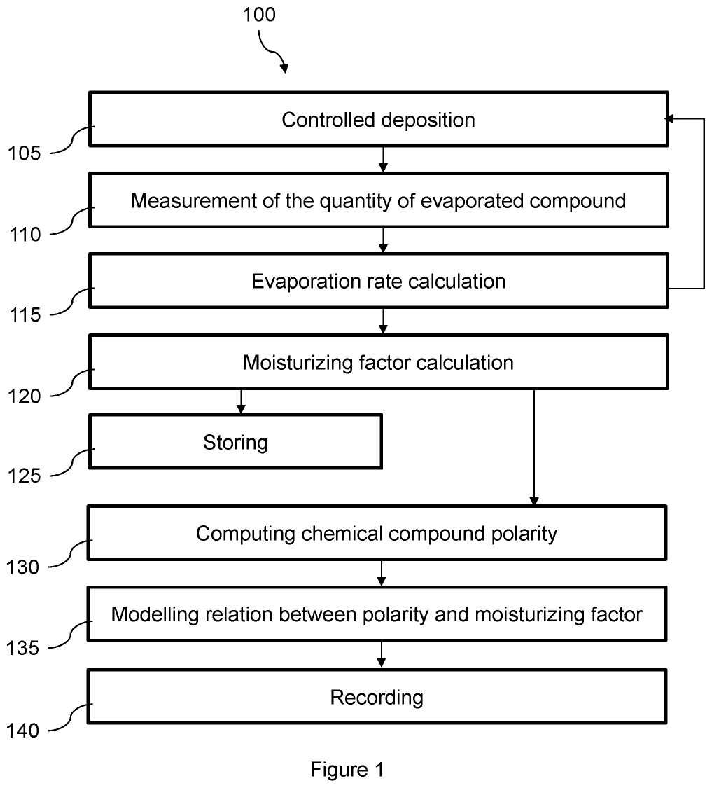

FIG. 1 shows a particular succession of steps of a method which is the subject of this invention. This volatile chemical compound physical parameter database construction method 100, comprises, at least:
a step 105 of controlled deposition of a fragrant chemical compound upon a skin replicating surface,
a step 110 of measurement of a quantity of evaporated water from the skin replicating surface or of remaining water on the skin replicating surface after the deposition of the fragrant chemical compound at different measurement times,
a step 115 of water evaporation rate calculation depending on the measured evaporated or remaining water quantities measured,
a step 120 of moisturizing factor calculation as a function of the water evaporation rate calculated and
a step 125 of storing, in a database, the calculated moisturizing factor and, optionally, the water evaporation rate calculated.

The step 105 of controlled deposition is performed, for example, by the transfer upon the skin replicating surface of a predetermined quantity of chemical compound set to spread over a predetermined surface. Such predetermination allows for the comparison of results as evaporation is in part due to the size surface of the compound in contact with the ambient environment. The more parameters are set and predetermined, the more accurate the water evaporation rate measurement is.

The transfer of the quantity of chemical compound can be performed using any known means to transfer liquids, preferably in small quantity, such as a pipette. Such a transfer can be performed manually or in an automatic manner.

The chemical compound considered can be in the form of a liquid or in the form of a solid diluted into a liquid. Preferably, such a chemical compound is pure. “Pure,” in this context, is intended as meaning “overwhelmingly containing said chemical compound.”

Rather than being adapted to single-compound analysis, the method 100 may be used upon fragrance compositions or mixtures, creating a database of compositions or mixture secondary benefit performance indicators.

This step 105 of controlled deposition is preferably performed at a controlled temperature and humidity throughout the evaporation quantity measurement.

Water evaporation rates or water content in the skin (in-vitro or in-vivo) are preferably measured in pseudo-equilibrium conditions: controlled temperature, air flow and rate, humidity to mimic a closed thermodynamic system.

The step 110 of measurement is performed, for example, using a Franz cell, whereby the skin replicating surface is placed within said cell, acting as the membrane, and the chemical compound sample to be analyzed is placed upon said surface. Measurement can typically be performed at the top of the Franz cell, above the membrane, which is different from the usual use of the Franz cell in which the measurements are made via a sampling port located below the membrane.

In other variants, any other type of measurement device may be used.

During this step 110 of measurement, the water loss from within the skin or skin model (in-vivo or in-vitro) to the external atmosphere or the water content remaining the skin or skin model is measured. In other embodiments, both are measured. Such measurements are preferably performed as function of time.

Such a measurement may be performed by a water evaporation quantity or water content measurement device (vapometer or moisturemeter) to produce values representative of Trans-Epidermal Water Loss (“TEWL”) or the water content (hydration).

Such measurements may be impacted by:
temperature,
surface of exposition upon the human skin mimic,
quantity of compound deposited,
air flow volume,
humidity, and
the composition or physical state of the supporting consumer product formulation such as solid soap bar or lotion in a form of emulsion

The step 115 of water evaporation rate calculation may be performed by running a computer software upon a computing device. Such a computing device may be unitary with a water evaporation quantity measurement device, for example. In other embodiments, such a computing device may be a computer or server associated to the water evaporation quantity measurement device. The calculated water evaporation rate may be such that said water evaporation rate is obtained by dividing the quantity of water evaporated measured by a value representative of a duration of evaporation. Depending on the characteristics of the system, such a duration may be 30 minutes, one hour or other such intervals. In particular embodiments, several water evaporation rates are computed for on chemical compound for different durations from chemical compound deposition.

In other embodiments, the calculated water evaporation rate may be such that said water evaporation rate is obtained by subtracting the quantity of remaining water from the initial quantity of water deposited and dividing the resulting quantity of water evaporated measured by a value representative of a duration of evaporation.

Such a water evaporation rate may be measured in quantity (absolute or relative) to be processed to produce a water evaporation rate. For example, a value of water evaporation rate may be given for a skin mimicking surface after deposition of a chemical compound losing 5% of the original deposited water quantity, due to evaporation, over 30 minutes. The water evaporation rate (g/m²h) can be calculated from the increase of relative humidity in function of time. Ambient temperature and humidity can be recorded using an external room sensor to account for environmental offset of the pending measurement.

The method 100 may further comprise a step of water evaporation reference quantity measurement a step of water evaporation reference rate calculation. Both these steps may be performed similarly to, or during, the step 110 of measurement and the step 115 of calculation disclosed above. In these steps, water replaces the fragrant chemical compounds, allowing for the definition of a reference value for evaporation rates comparison.

The step 120 of moisturising factor calculation may be performed, for example, by running a computer software upon a computing device. During this step 120 of moisturising factor calculation, the following formula may be implemented:

MF=A−B/A×100

### Wherein:

MF represents the moisturising factor,
A represents the evaporation rate of the water from a reference (skin or skin replicating surface) before deposition of the investigated fragrant chemical compound (or mixture) and
B represents the evaporation rate of the water from the skin or a skin replicating surface after the deposition of the examined fragrant chemical compound (or fragrant mixture).

If the moisturising factor is zero, the behaviour of the chemical compound is equal to the reference and means that the raw material is non-moisturising. If the moisturising factor is 100, a maximum of moisturization is obtained. A chemical compound is considered as moisturising when its moisturising factor is equal or higher than 1.

Generally, the “moisturising factor” designates any metric representative of the capacity of a chemical compound to retain water upon the human skin. Many variants of the formula above may be selected to compare such a performance for chemical compounds.

The step 125 of storing is performed, for example, by a computerized database accessible by the computing means configured to perform the water evaporation rate computing. Such a database can be stored upon a server, for example.

During the step 125 of storing, any value representative of the capacity of the volatile compound of moisturizing the human skin may be stored, such as the moisturizing factor associated to said volatile compound or the polar energy density of said volatile compound.

In particular embodiments, the method 100 object of the present invention comprises:
a step 130 of computing a chemical compound polarity for a fragrant chemical compound associated to a stored moisturizing factor,
a step 135 of modeling of a mathematical formula of moisturizing factor as a function of chemical compound polarity and
a step 140 of recording, in a database, the moisturizing factor formula modeled parameters.

Such a compound polarity should be understood as the polar energy density, such as shown in the equation below.

The step 130 of computing is performed, for example, by running a computer program upon a computing device. During this step 130 of computing, the following mathematical formula may be computed:

E*_{P}=∂_{Pol}²=∂_{P}²+δ_{H}²

### Wherein:

E*_{P} designates the polar energy density of a chemical compound,
δ_{Pol}² designates the polar energy density of a chemical compound, expressed in megapascals,
δ_{P}² designates the polar bonding component of the cohesion energy density of a chemical compound, expressed in megapascals, where δ_{P} is known as the polar Hansen parameter and
δ_{H}² designates the hydrogen bonding component of the cohesion energy density of a chemical compound, expressed in megapascals, where δ_{H} is known as the hydrogen-bonding Hansen parameter.

In particular embodiments, the fragrant chemical compound polarity is computed as a function of at least one of the dispersion, polar and hydrogen bonding components of the cohesion energy density, related to the dispersion, polar and hydrogen bonding interactions of said fragrant chemical compound.

In other embodiments, the polar energy density of a chemical compound is obtained by retrieving said value from a database associating polar energy density to chemical compound digital identifiers.

The step 135 of modeling is performed, for example, by running a computer program upon a computing device. Such a modeling intends to perform a fit between a mathematical formula and the computed moisturizing factor and the calculated polar energy density. Such a mathematical formula can be a logarithmic curve or exponential equation for example in which parameters are set to match the moisturizing factor as a function of the polar energy or cohesive energy density.

Such a logarithmic curve can be seen in FIG. 5 which shows:
a y-axis representative of increasing computed moisturizing factor,
an x-axis representative of increasing polar energy density and
a logarithmic curve fitting the distribution.

The parameters of said curve, or mathematical formula, are then recorded in a database during the step 140 of recording.

In particular embodiments, the modeled moisturizing factor is a function of a logarithm or exponential function of the chemical compound polarity.

In other embodiments, during the step 135 of modeling, the correlation between moisturizing factor and the hydrogen bonding component of the cohesion energy density of a chemical compound may be performed.

In other embodiments, during the step 135 of modeling, the correlation between moisturizing factor and the polar bonding component of the cohesion energy density of a chemical compound may be performed.

In other embodiments, during the step 135 of modeling, the correlation between moisturizing factor and sum of the polar bonding component of the cohesion energy density and the hydrogen bonding component of the cohesion energy density of a chemical compound may be performed.

In other embodiments, during the step 135 of modeling, the correlation between moisturizing factor and cohesive energy density of a chemical compound may be performed.

This step 140 of recording is performed, for example, in a similar manner to the step 125 of storing.

In another embodiment of the present invention, not represented in the drawings, the method object of the present invention comprises:
a step of retrieving, from a database, a value representative of a moisturizing factor associated to at least one fragrant chemical compound identifier,
a step of retrieving, from a database, a value representative of a polar energy density associated to at least one fragrant chemical compound identifier,
a step of modeling of a mathematical formula of moisturizing factor as a function of chemical compound polarity and
a step of storing at least one mathematical formula parameter in a database.

FIG. 8 shows, schematically, a particular succession of steps of the method 800 object of the present invention. The volatile chemical compound physical parameter database construction method 800 comprises, at least:
a step 805 of measurement or calculation of a compound polarity,
a step 810 of moisturizing factor calculation as a function of the measured or calculated compound polarity and
a step 815 of storing, in a database, the calculated moisturizing factor and, optionally, the compound polarity measured or calculated.

The step 805 of measurement may be performed in a similar manner to the step 130 of computing.

The step 810 of moisturizing factor calculation may be performed in a similar manner to the step 135 of modelling.

The step 815 of storing may be performed in a similar manner to the step 125 of storing.

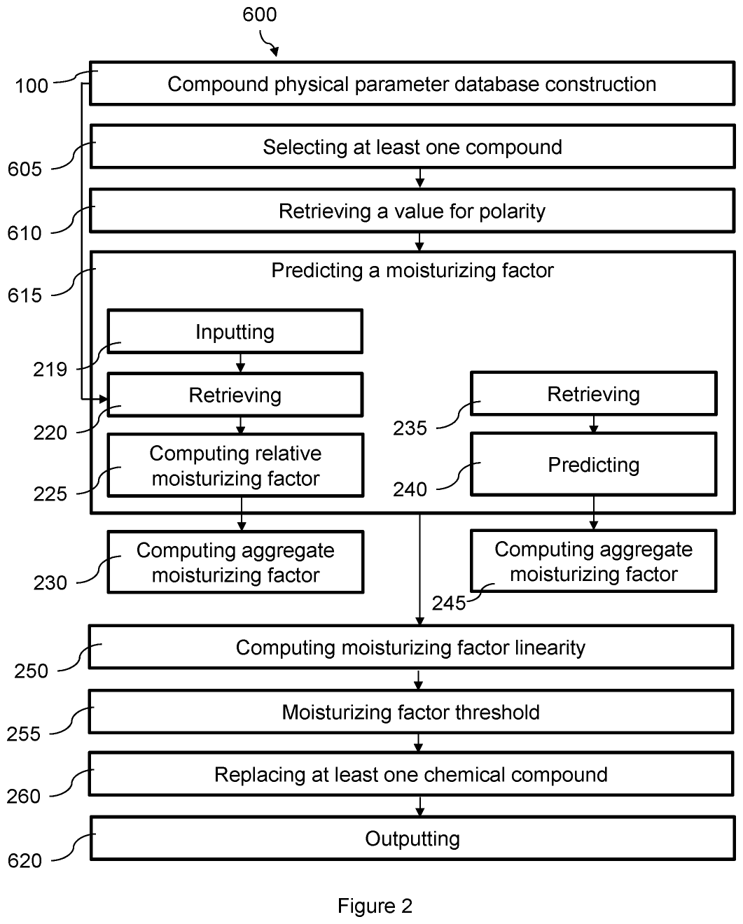

FIG. 2 represents, schematically, a particular embodiment of the method 600 object of the present invention. This computer implemented method 600 to provide predictive, real time, skin hydration performance metrics for a composition, comprises, at least:
a step 605 of selecting at least one fragrant chemical compound identifier upon a computerized interface,
a step 615 of predicting a moisturizing factor associated to at least one fragrant chemical compound identifier and
a step 620 of outputting at least one mean moisturizing factor obtained.

The step 605 of selecting may be performed by any means of inputting. The means of inputting is, for example, a keyboard, mouse and/or touchscreen adapted to interact with a computing system in such a way to collect user input. In variants, the means of inputting are logical in nature, such as a network port of a computing system configured to receive an input command transmitted electronically. Such an input means may be associated to a GUI (Graphic User Interface) shown to a user or an API (Application programming interface). In other variants, the means of inputting may be a sensor configured to measure a specified physical parameter relevant for the intended use case.

In some embodiment, a user may select upon at least one fragrant chemical compound digital identifier upon GUI. In more perfected embodiments, the user may create a fragrance formula by selecting at least one fragrant chemical compound digital identifier. Said formula is, for example, representative of a fine perfume to be manufactured.

The step 615 of predicting a moisturizing factor may be performed in a variety of ways, depending on whether computation is required at this step.

In a first embodiment, the step 615 of predicting comprises a step 220 of retrieving, from a database constituted according to the method object of the present invention, a moisturizing factor associated to at least one fragrant chemical compound identifier. Such a moisturizing factor may then be output upon a computer interface.

Such a step 220 of retrieving may be performed, for example, by running a computer software upon a computing device. During this step 220 of retrieving, a database associating fragrant chemical compound digital identifiers to values representative of moisturizing factor is accessed and using at least one selected fragrant chemical compound digital identifier as a search key within the database, at least one corresponding moisturizing factor is retrieved.

In a second embodiment, the step 615 of predicting comprises:
a step 235 of retrieving, from a database, at least one value representative of the polarity of said fragrant chemical compound identifier,
a step 240 of predicting at least one moisturizing factor for at least one fragrant chemical compound identifier as a function of at least one value representative of the polarity of said fragrant chemical compound identifier
   the step 215 of outputting being configured to output at least one moisturizing factor predicted.

Such a step 235 of retrieving may be performed, for example, by running a computer software upon a computing device. During this step 235 of retrieving, a database associating fragrant chemical compound digital identifiers to values representative of polarity is accessed and using at least one selected fragrant chemical compound digital identifier as a search key within the database, at least one corresponding polarity is retrieved.

In variants, during the step 235 of retrieving, values representative of dispersion, polar and hydrogen bonding components of the cohesion energy density, related to the dispersion, polar and hydrogen bonding interactions are retrieved. In such embodiments, the chemical compound polarity or cohesive energy density is calculated during a downstream step of polarity or cohesive energy density calculation. Such a polarity or cohesive energy density calculation may be similar to the step 130 of computing disclosed above.

The step 240 of predicting may be performed, for example, by running a computer software upon a computing device. During this step 240 of predicting, the retrieved or computed polarity value is used in a mathematical formula modeling the relationship between polarity and moisturizing factor to obtain a predicted, or inferred, moisturizing factor value for a determined chemical compound.

This moisturizing factor may then be output during the step of outputting 215.

The step 215 of outputting may be performed in a similar, albeit reversed, manner to the step 205 of selecting. During this step 215 of outputting, a means for output of a computing device may be used, such as a computer screen or network port. Digital means of output, such as APIs may also be used.

For example, the step 215 of outputting may use a GUI in which each selected chemical compound digital identifier forming a fragrance formula is associated to a displayed moisturizing factor. The moisturizing factor may be shown, for example, as an alphanumeric label or as an icon varying according to the value of the moisturizing factor.

Such GUI may provide advanced capabilities to users, such as the capacity to modify a designed formula on the basis of the moisturizing factor predicted or computed.

In one such advanced embodiment, the method 200 comprises:
a step 219 of inputting, for each selected chemical compound, a quantity of said chemical compound and/or
a step 225 of computing a mean moisturizing factor for at least one fragrant chemical compound identifier as a function of the moisturizing factor retrieved or predicted and the quantity input for said fragrant chemical compound identifier and
   the step 215 of outputting being configured to output at least one mean moisturizing factor computed.

The step 219 of inputting may be performed, structurally, with similar means to the step 205 of selecting. The quantity set may be in relative proportion, within the fragrance formula, or absolute quantity. In an example, a user may use a keyboard to input a relative proportion of a chemical compound in a formula within a dedicated GUI displayed upon a computer screen linked to a computing device.

The step 225 of computing may be performed, for example, by running a computer software upon a computing device. During this step 225 of computing, the moisturizing factor may be mathematically weighted according to the relative proportion in quantity of each said chemical compound. This allows for the visualization of the impact on overall moisturization capacity, for a fragrance formula, of the constituting chemical compounds of that formula.

In particular embodiments, at least two fragrant chemical compound identifiers are selected, the method further comprising a step 230 of computing a composition moisturizing factor as a function of at least two mean moisturizing factors computed.

The step 230 of computing may be performed, for example, by running a computer software upon a computing device. During this step 230 of computing, an average or weighted average of moisturizing factors of constituting chemical compounds may be calculated in order to form a composition moisturizing factor.

In particular embodiments, at least two fragrant chemical compound identifiers are selected, the method further comprising a step 245 of computing an aggregate predicted moisturizing factor as a function of at least two mean moisturizing factors computed.

Such a step 245 may be performed similarly to the step 230 of computing.

In particular embodiments, at least two chemical compound identifiers are selected, the method further comprising a step 250 of computing an moisturizing factor linearity of a composition of said at least two compound identifiers based on the obtained moisturizing factor of at least two selected chemical compound digital identifiers, the step 215 of outputting being configured to display the moisturizing factor linearity of the composition of said at least two chemical compound digital identifiers.

In particular embodiments, the moisturizing factor of a mixture or composition of fragrant chemical compounds can be calculated in the following manner:
determining the polar energy density of the composition:

E*ₚ(mixture)=Σᵢxi*E*ᵢ(compound)

where “xi” corresponds to the quantity of the chemical compound in the composition and “i” being the number of the chemical compounds in the composition and
determining the moisturizing factor of the composition:

MF(mixture)=f(E*ₚ(mixture))

where function “f” corresponds to the modelled mathematical formula linking polar energy density to moisturizing factor.

Such embodiments may also be adapted to calculations based upon subcomponents of polar energy density, such as disclosed above.

The step 250 of computing may be performed, for example, by running a computer software upon a computing device. During this step 250 of computing, a statistical indicator, such as mean or standard deviation, may be computed for each moisturizing factor of each chemical compound constituting a fragrance formula. A lower standard deviation means greater chemical compound coherency in terms of moisturization effect, for example.

In particular embodiments, the method 200 object of the present invention further comprises a step 255 of defining a moisturizing factor threshold, at least one chemical compound digital identifier being removed from the selection as a function of the difference between the moisturizing factor being obtained for said chemical compound digital identifier and the defined moisturizing factor threshold.

The step 255 of defining may be performed, structurally, with similar means to the step 205 of selecting. The threshold is defined as a numerical value, for example, representing a moisturizing factor defining an applicability domain for the fragrance created.

The step of removal of at least one chemical compound digital identifier may be triggered if at least one said chemical compound digital identifier presents a moisturizing factor below or above the threshold, depending on the intended cosmetic application of fragrance.

The step of removal may be performed, for example, by running a computer software upon a computing device executing the above-mentioned check.

In particular embodiments, the method 200 object of the present invention further comprises a step 260 of replacing at least one chemical compound digital identifier as a function of the difference between the moisturizing factor being obtained for said chemical compound digital identifier and the moisturizing factor being obtained for an alternative chemical compound digital identifier candidate.

The step 260 of replacing may be performed, for example, by running a computer software upon a computing device. During this step 260 of replacing, a chemical compound candidate for replacement is selected, either manually or automatically. Another chemical compound is searched for, in a database, and selected, provided this chemical compound is associated to a higher or lower moisturizing factor (depending on the intended use case). Such a replacement may be suggested to the user of the system or automatically replace the chemical compound candidate for replacement.

A multi-criteria approach may be undertaken wherein candidates to replace a chemical compound are selected based upon moisturizing factor as well as other factors, such as fragrance tonality, for example.

FIG. 5 represents, schematically, a particular embodiment of the method 500 object of the present invention. This fragrance composition assembling method, comprises:
a fragrance physical composition prediction method 600 such as disclosed in regard to FIG. 2 and
a step 505 of assembling the prediction fragrance composition.

The step 505 of assembling may be performed according to any fragrance manufacturing process known to a person skilled in the art that is relevant for the selected composition of chemical compounds.

As it is understood, the present invention also aims at fragrance composition, obtained according to a fragrance composition assembling method such as disclosed in regard to FIG. 5.

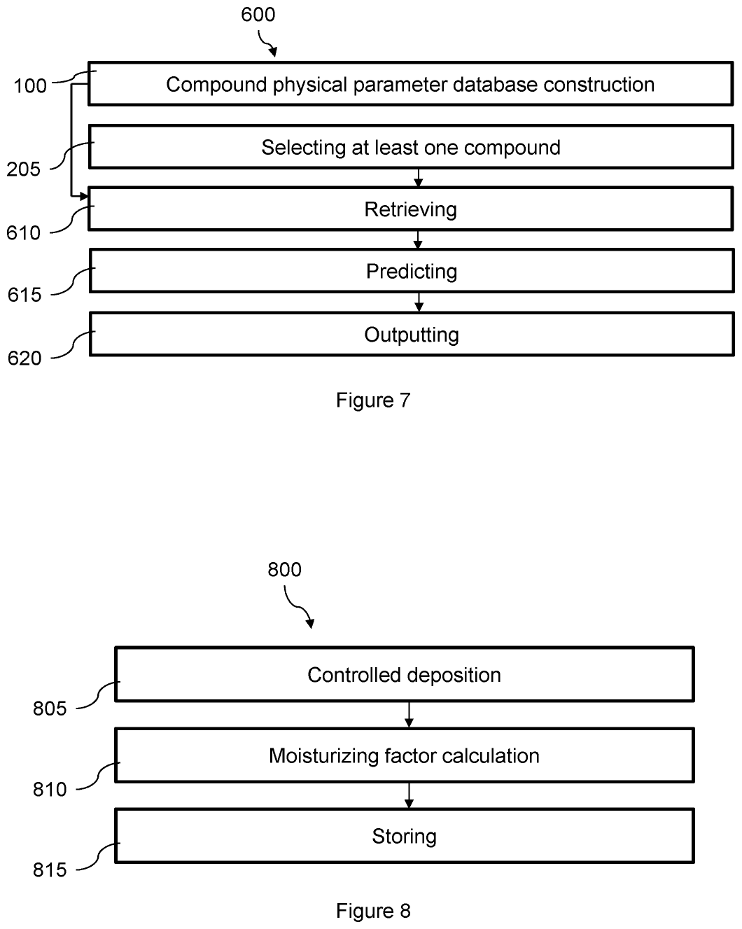

FIG. 7 shows, schematically, a particular embodiment of the method 600 object of the present invention. Computer implemented method 600 to provide predictive, real time, skin hydration performance metrics for a composition comprises, at least:
a step 205 of selecting, upon a computer interface, at least one fragrant chemical compound digital identifier, to form a composition,
a step 610 of retrieving, from a database, at least one value representative of a polarity value of at least one selected chemical compound identifier,
a step 615 of predicting at least one moisturizing factor value for at least one chemical compound identifier or of the composition as a function of at least one retrieved polarity value and
a step 620 of outputting at least one moisturizing factor value predicted.

The step 205 of selecting may be performed in a similar manner to the step 205 of selected described in regard to FIG. 2.

The step 610 of retrieving may be performed in a similar manner to the step 220 of selected described in regard to FIG. 2.

The step 615 of predicting may be performed in a similar manner to the step 240 of predicting described in regard to FIG. 2.

The step 620 of outputting may be performed in a similar manner to the step 215 of outputting described in regard to FIG. 2.

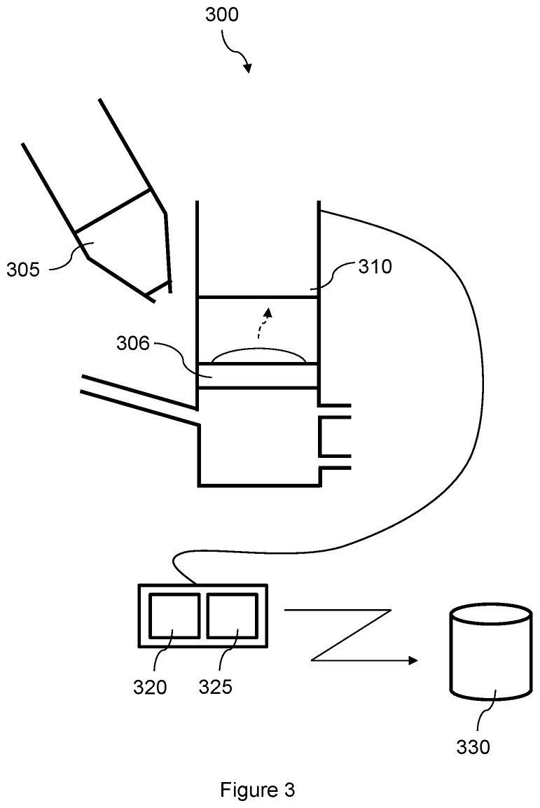

FIG. 3 shows, schematically, a particular embodiment of the system 300 object of the present invention. This volatile liquid chemical compound physical parameter database construction system 300 comprises, at least:
a means 305 of controlled deposition of a fragrant chemical compound upon a skin replicating surface,
a means 310 of measurement of a quantity of evaporated water from the skin replicating surface or of remaining water on the skin replicating surface after the deposition of the fragrant chemical compound at different measurement times,
a means 315 of water evaporation rate calculation depending on the measured evaporated or remaining water quantities measured,
a means 320 of moisturizing factor calculation as a function of the water evaporation rate calculated and
a means 325 of storing, in a database 330, the calculated moisturizing factor and, optionally, the water evaporation rate calculated.

Examples of embodiments of such means are disclosed with regard to the corresponding methods.

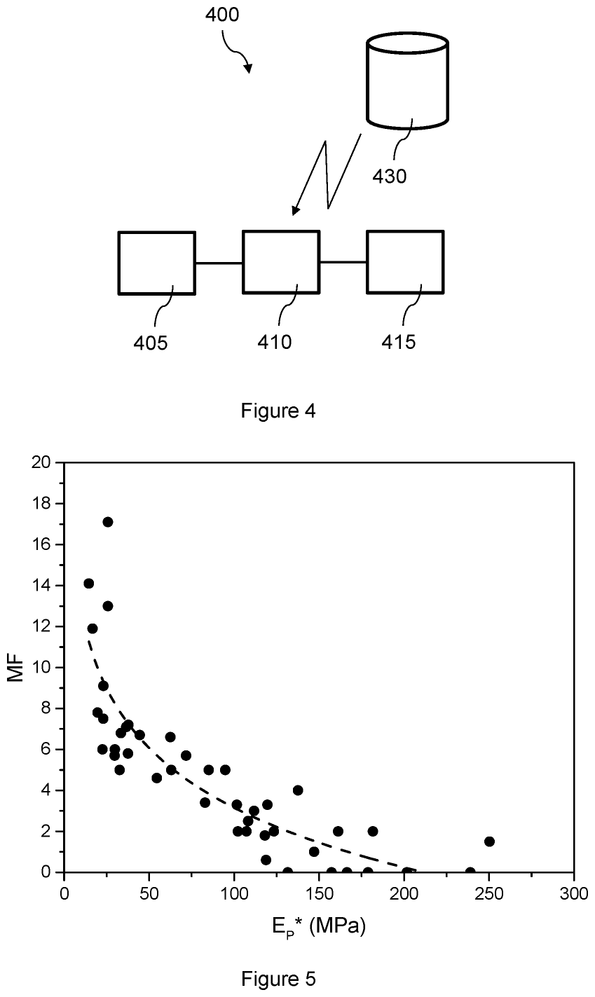

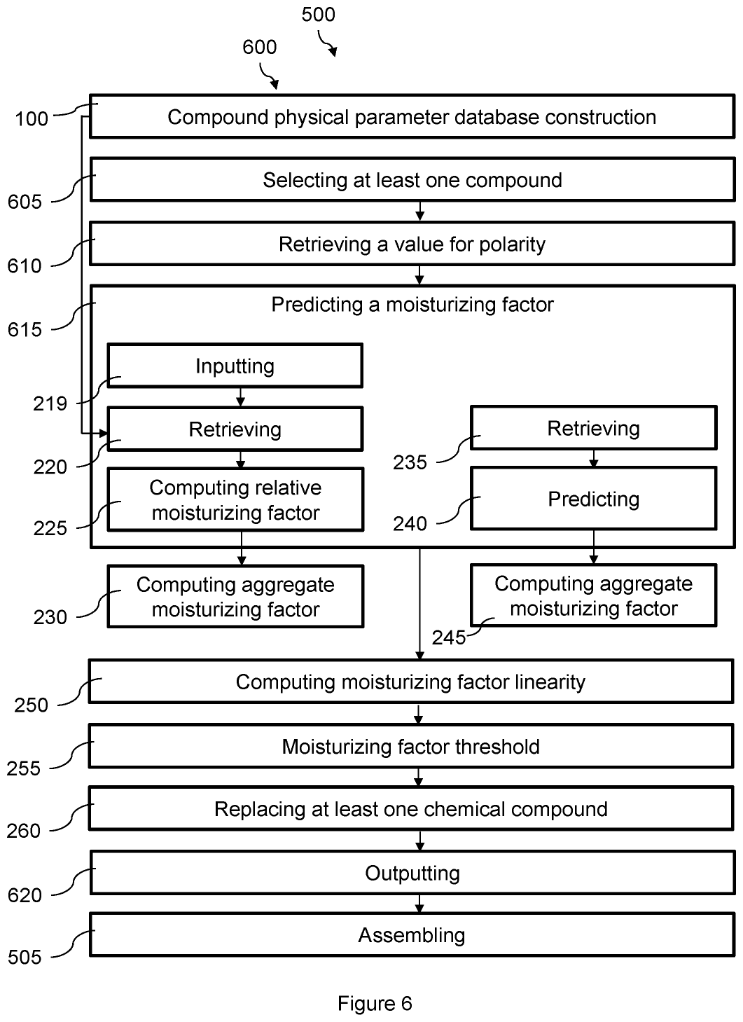

FIG. 4 shows, schematically, a particular embodiment of the system 400 object of the present invention. This fragrance physical composition prediction system 400 to provide predictive, real time, secondary cosmetic benefits fragrance performance metrics comprises, at least:
a means 405 of selecting at least one fragrant chemical compound identifier upon a computerized interface,
a means 410 of obtaining a moisturizing factor associated to at least one fragrant chemical compound identifier and
a means 415 of outputting at least one mean moisturizing factor obtained.

Examples of embodiments of such means are disclosed with regard to the corresponding methods.

1. Computer implemented method to provide predictive, real time, skin hydration performance metrics for a composition, comprising, at least:
a step of selecting, upon a computer interface, at least one chemical compound digital identifier, to form a composition,
a step of retrieving, from a database, at least one value representative of a polarity value of at least one selected chemical compound identifier,
a step of predicting at least one moisturizing factor value for at least one chemical compound identifier or of the composition as a function of at least one retrieved polarity value and
a step of outputting at least one moisturizing factor value predicted.

2. Method according to claim 1, which comprises a chemical compound physical parameter database construction step, comprising at least:
a step of controlled deposition of a volatile chemical compound, or a non-volatile compound in a liquid state, upon a skin replicating surface,
a step of measurement of a quantity of evaporated water from the skin replicating surface or of remaining water on the skin replicating surface after the deposition of the chemical compound at different measurement times,
a step of water evaporation rate calculation depending on the measured evaporated or remaining water quantities measured,
a step of moisturizing factor calculation as a function of the water evaporation rate calculated and
a step of storing, in a database, the calculated moisturizing factor and, optionally, the water evaporation rate calculated.

3. Method according to claim 1, which further comprises:
a step of computing a chemical compound polarity for a chemical compound associated to a stored moisturizing factor,
a step of modelling of a mathematical formula of moisturizing factor as a function of chemical compound polarity and
a step of recording, in a database, the moisturizing factor formula modelled parameters.

4. Method according to claim 3, in which the chemical compound polarity is computed as a function of at least one of the dispersion, polar and hydrogen bonding components of the cohesion energy density, related to the dispersion, polar and hydrogen bonding interactions of said chemical compound.

5. Method according to claim 3, which comprises a step of inputting, for each selected chemical compound, a quantity of said chemical compound, the step of obtaining comprising a step of computing a mean moisturizing factor for at least one chemical compound identifier as a function of the moisturizing factor and the quantity input for said chemical compound identifier, the step of outputting being configured to output at least one mean moisturizing factor computed.

6. Method according to claim 5, in which at least two chemical compound identifiers are selected, the method further comprising a step of computing a composition mean moisturizing factor as a function of at least two moisturizing factors computed.

7. Method according to claim 6, in which at least two chemical compound identifiers are selected, the method further comprising a step of computing a composition moisturizing factor as a function of at least two mean moisturizing factors predicted.

8. Method according to claim 1, in which at least two chemical compound identifiers are selected, the method further comprising a step of computing an moisturizing factor linearity of a composition of said at least two compound identifiers based on the predicted moisturizing factor of at least two selected chemical compound digital identifiers, the step of outputting being configured to display the moisturizing factor linearity of the composition of said at least two chemical compound digital identifiers.

9. Method according to claim 1, which further comprises a step of defining a moisturizing factor threshold, at least one chemical compound digital identifier being removed from the selection as a function of the difference between the moisturizing factor being obtained for said chemical compound digital identifier and the defined moisturizing factor threshold.

10. Method according to claim 1, which further comprises a step of replacing at least one chemical compound digital identifier as a function of the difference between the moisturizing factor being obtained for said chemical compound digital identifier and the moisturizing factor being obtained for an alternative chemical compound digital identifier candidate.

11. Method of chemical compound composition assembly, comprising:
a composition prediction method according to claim 1 and
a step of assembling the composition object of the prediction method.

12. Composition, characterized in that it is obtained according to a composition assembling method according to claim 11.

13. Method according to claim 1, in which at least one chemical compound is a fragrant chemical compound.
